(19) United States
(12) Patent Application Publication
(10) Pub. No.: US 2026/0034058 A1
(43) Pub. Date: Feb. 5, 2026

(54) FREEZE-DRIED BITE WITH SINGLE DOSE PSYCHEDELIC

(71) Applicant: VITALMELT LTD., Petah Tikva (IL)

(72) Inventor: David KLIGER, Ramat Gan (IL)

(21) Appl. No.: 19/101,442

(22) PCT Filed: Jul. 24, 2023
(86) PCT No.: PCT/IL2023/050772

§ 371 Date: Feb. 5, 2025

Related U.S. Application Data
(60) Provisional application No. 63/396,333, filed on Aug. 9, 2022.

(51) Int. Cl. A61K 9/00; A61K 9/19; A61K 31/135; A61K 31/36; A61K 31/48; A61K 31/675; A61K 47/44; A61K 47/46

(52) U.S. Cl. CPC A61K 9/0056; A61K 9/19; A61K 31/135; A61K 31/36; A61K 31/48; A61K 31/675; A61K 47/44; A61K 47/46

(57) ABSTRACT

Provided herein are freeze-dried psychedelic compositions including a mixture of fruit and/or vegetable puree; an edible oil; and a psychedelic; wherein the freeze-dried psychedelic composition is a single dose composition that melts in the mouth.

### TECHNOLOGICAL FIELD

The present invention provides a freeze-dried solid psychedelic composition in a product form that instantly melts in the mouth, processes for its preparation.

### BACKGROUND

Psychedelics are a group of compounds that have received increasing attention, both for their recreation and medicinal properties. In particular, micro-dosing with psychedelics has been shown to have a beneficial effect on many mental illnesses, improving mood, reducing anxiety and enhancing focus.

For example, depression results in reduced appetite, and paranoia in disorders such as schizophrenia, may result in a reluctance to take tablets and/or pills. Additionally, many people find swallowing pills and/or capsules difficult. Moreover, many recreational drugs are cut with toxic substances or uncontrolled amounts of additional excipients or may be tampered with.

Accordingly, there is a need for an improved psychedelics composition, which can provide various psychedelics in a dosage from which need not be swallowed, which manufactured by a trustworthy source in a tamper resistant form, and which encourages compliance.

### SUMMARY

According to some embodiments, provided herein are advantageous freeze-dried solid psychedelic composition in a product form that can instantly melt in the mouth of a subject. According to some embodiments, the psychedelic composition may include fruit and/or vegetable puree, edible oil and at least about one psychedelic compound, wherein after mixing such components, the composition is freeze dried, to the ready to be consumed psychedelic composition product.

In some embodiments, the present invention provides a psychedelic product/composition which may include: at least about 10% w/w of a fruit puree; at least about 4% w/w of an edible oil; and about 0.000005% w/w-about 10% w/w of a pure psychedelic compound (or combination of psychedelic compounds); wherein the psychedelic composition is freeze dried into a ready to be consumed product form which is capable of melting in the mouth.

Advantageously, as exemplified herein, the components of the psychedelic product may maintain at least about 75%, even at least about 90% and even at least about 95% of their initial psychedelic value (i.e., prior to freeze drying).

As a further advantage, as further exemplified herein, the psychedelic product may be devoid of synthetic preservatives. Advantageously, all ingredients in the psychedelic product may be 100% natural. Advantageously, at least one of the components of the psychedelic product may be organic.

Additionally, according to some embodiments, the composition of the present invention does not have to be further prepared in any manner. It does not have to be cooked, rehydrated, steamed, mixed with a liquid (hot or cold), fried, microwaved or any other form of preparation. A product of the present invention is ready to be consumed by a subject as is, without any other additions or preparations. Thus, the composition of the present invention is readily consumed while performing other tasks such as working, driving, walking, etc.

According to some embodiments, the composition may be a solid porous matrix (non-powdered). According to some embodiments, the composition may have the form of a single bite. According to some embodiments, the volume of the single bite may be at least about 4 cm³, at least about 5 cm³, at least about 6 cm³, or at least about 8 cm³. Each possibility is a separate embodiment. According to some embodiments, the volume of the single bite may be between about 4 cm³ and about 8 cm³ or about 4 cm³ and about 6 cm³. Each possibility is a separate embodiment.

According to some embodiments, advantageously, the ready to consume psychedelic product disclosed herein may have a good taste, which may encourage compliance when such is prescribed and/or desired.

According to some embodiments, due to the product being capable of melting in the mouth, the hereindisclosed compositions may be used and/or consumed by a wide range of subjects, including, healthy subjects, and subjects afflicted with a disease, disorder or condition. In some embodiments, the hereindisclosed compositions may be particularly useful for administration to subject's having trouble swallowing, such as for example, but not limited to bariatric patient.

According to some embodiments, there are provided herein advantageous psychedelic compositions, which are ready for oral consumption, cost efficient and effective in providing various psychedelic compounds to subjects, for preventing and/or improving health condition of subjects (users) and/or enjoyment in consuming the product. In some embodiments, the compositions disclosed herein may be advantageous, as they may exhibit improved bioavailability, pharmacokinetics and/or pharmacodynamics of psychedelic substances included within the compositions, thereby facilitating an enhanced effect thereof.

According to some embodiments, there is provided a psychedelic composition which may include a mixture of: (a) at least about 10% w/w of a fruit and/or vegetable puree; (b) at least about 4% w/w of an edible oil; and (c) at least about 0.000005% w/w of a psychedelic compound (which may include one or more psychedelic compounds), or of a mixture which of a psychedelic compound and a thickening agent.

According to some embodiments, the psychedelic composition may be freeze dried to obtain a ready to be consumed product form, which may be capable of melting in the mouth.

According to some embodiments, the composition may include less than about 5% w/w water.

According to some embodiments, the composition may include at least about 15% w/w-about 70% w/w of the fruit and/or vegetable puree. In some embodiments, the composition may include at least about 15% w/w-about 70% w/w of fruit puree. In some embodiments, the composition may include at least about 15% w/w-about 70% w/w of vegetable puree. According to some embodiments, the fruit puree and/or the vegetable puree may be organic.

According to some embodiments, the composition may include about 4%-about 40% w/w of an edible oil. According to some embodiments, the edible oil may be organic.

According to some embodiments, the composition may include at least about 0.000005% w/w of a psychedelic compound. According to some embodiments, the composition may include at least about 0.000003% w/w of a psychedelic compound. According to some embodiments, the composition may include at least about 0.000002% w/w of a psychedelic compound. According to some embodiments, the composition may include at least about 0.000002% w/w of a psychedelic compound. According to some embodiments, the composition may include at least about 0.000001% w/w of a psychedelic compound. According to some embodiments, the composition may include between 0.000001%-10% w/w of a psychedelic compound.

According to some embodiments, the psychedelic compound may be organic. According to some embodiments, the psychedelic compound may be a natural extract or compound, a derivative of a natural compound, a synthetic compound, or a mixture of more than one compound. For example, the psychedelic compound may be psilocin, psilocybin, ketamine, ergoline, lysergamide (e.g., lysergic acid diethylamide (LSD), methamphetamine (e.g., 3,4-methylenedioxy methamphetamine (MDMA)), mescaline, ibogaine, tryptamine (e.g., N,N-dimethyltryptamine (DMT)), phenethylamine, N-methoxybenzyl compound, 4-bromo-2,5-dimethoxyphenethylamine, myristicin, benzoxazine, beta-carboline, entheogen, peyote, serotonergic hallucinogen, etc. and/or derivatives, metabolites, isomers, stereoisomers, or combinations thereof. Each possibility is a separate embodiment.

According to some embodiments, the psychedelic compound may be a plant, or part thereof. For example, the plant may be a fungus (e.g., psilocybin mushroom, etc.), cactus (e.g., peyote cactus, etc.), herbaceous plant (e.g., Salvia divinorum, etc.), flowering plant (e.g., various morning-glory species such as Turbina corymbose, datura species, etc.), and/or combinations thereof. Each possibility is a separate embodiment. According to some embodiments, the psychedelic compound may be part of a plant, such as a root, stem, leaf, seed, flower, and/or fruit.

According to some embodiments, the composition may include at least about 0.000005% w/w of the psychedelic compound and about 15% w/w-about 99% w/w of a fruit puree and/or a vegetable puree.

According to some embodiments, the psychedelic compound may be a fungus. According to some embodiments, the composition may include at least about 0.05% w/w, at least about 0.5% w/w, at least about 5% w/w, at least about 10% w/w, at least about 15% w/w, at least about 20% w/w, at least about 25% w/w, or at least about 30% w/w, at least about 40% w/w, or at least about 50% w/w of the fungus. According to some embodiments, the fungus may be a psilocybin mushroom.

According to some embodiments, the psychedelic compound may be or may include psilocybin, LSD, MDMA and/or ketamine. Each possibility is a separate embodiment.

According to some embodiments, the composition may comprise about 1-5 g dried psilocybin mushroom or about 10-50 mg isolated psilocybin. According to some embodiments, the composition may comprise about 10-70% w/w dried psilocybin mushroom or about 0.01-1% w/w isolated psilocybin.

According to some embodiments, the composition may comprise about 10-400 μg LSD. According to some embodiments, the composition may comprise about 0.000005% w/w-about 0.0001% w/w LSD.

According to some embodiments, the composition may comprise about 10-500 mg MDMA. According to some embodiments, the composition may comprise about 0.001% w/w-about 10% w/w MDMA.

According to some embodiments, the composition may comprise about 30-200 mg ketamine. According to some embodiments, the composition may comprise about 0.002% w/w-about 5% w/w ketamine.

According to some embodiments, the composition may include a flavoring agent. According to some embodiments, at least one flavoring agent may be organic.

According to some embodiments, the composition may include a sweetener. According to some embodiments, the composition may include at least about 15% w/w of the sweetener. According to some embodiments, the composition may include about 20% w/w-about 40% w/w of the sweetener. According to some embodiments, at least one sweetener may be organic.

According to some embodiments, the composition may include an edible oil. According to some embodiments, the edible oil may be organic. According to some embodiments, the edible oil may be an oil having a melting temperature below about 50° C. According to some embodiments, the edible oil may be an oil having a melting temperature below about 40° C. According to some embodiments, the edible oil may be selected from a vegetable oil, a saturated fatty acid, unsaturated fatty acid, or any combinations thereof. According to some embodiments, the edible oil may be coconut oil. According to some embodiments, the composition may include about 10% w/w-about 40% w/w of the edible oil.

According to some embodiments, the composition may include about 1-5% w/w of water. According to some embodiments, the composition may include less than about 5% w/w of water.

According to some embodiments, there is provided a process for the preparation of a freeze-dried psychedelic composition, the process includes the steps of:
a. mixing a psychedelic compound with an edible oil to obtain a first mixture, wherein the mixing is performed at a temperature of below about 50° C.;
b. mixing the first mixture with a fruit and/or vegetable puree to obtain a second mixture;
c. freeze drying said second mixture until water content is less than about 5% w/w of the product; thereby producing a freeze-dried psychedelic composition having a ready to be consumed product form, which melts in the mouth.

According to some embodiments, the psychedelic compound may be encapsulated.

According to some embodiments, step (a) includes mixing at least about 0.000005% w/w-about 10% w/w of the final product of the psychedelic compound with at least about 4% w/w of the final product of the edible oil.

According to some embodiments, step (b) includes mixing the first mixture with at least about 10% w/w of the final product of the fruit and/or vegetable puree.

According to some embodiments, the method may further include adding a flavoring agent and/or a sweetener at step (a) and/or at step (b). According to some embodiments, the method may further include adding a flavoring agent and/or a sweetener at step (b). According to some embodiments, at least about 15% w/w of the final product of sweetener may be added.

According to some embodiments, the second mixture is cast into a mold prior to the freeze drying. According to some embodiments, the second mixture is frozen and shaped prior to freeze drying.

According to some embodiments, there is provided a process for the preparation of a freeze-dried psychedelic composition, the process includes the steps of mixing a psychedelic compound with a vegetable and/or fruit puree and edible oil, at a temperature of below about 50° C. and freeze drying said mixture until water content is less than about 5% w/w of the product.

According to some embodiments, there is provided a method for administering a psychedelic compound to a subject, the method comprising inserting into the subject's mouth, the psychedelic composition, as disclosed herein.

According to some embodiments, there is provided psychedelic composition, as essentially disclosed herein, for use in administration of a psychedelic compound to a subject in need thereof.

According to some embodiments, the subject is a subject has difficulty chewing or swallowing, undergoing or having undergone bariatric surgery.

Certain embodiments of the present disclosure may include some, all, or none of the above advantages. One or more technical advantages may be readily apparent to those skilled in the art from the Figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The invention will now be described in relation to certain examples and embodiments with reference to the following illustrative figures so that it may be more fully understood.

### In the Figures:

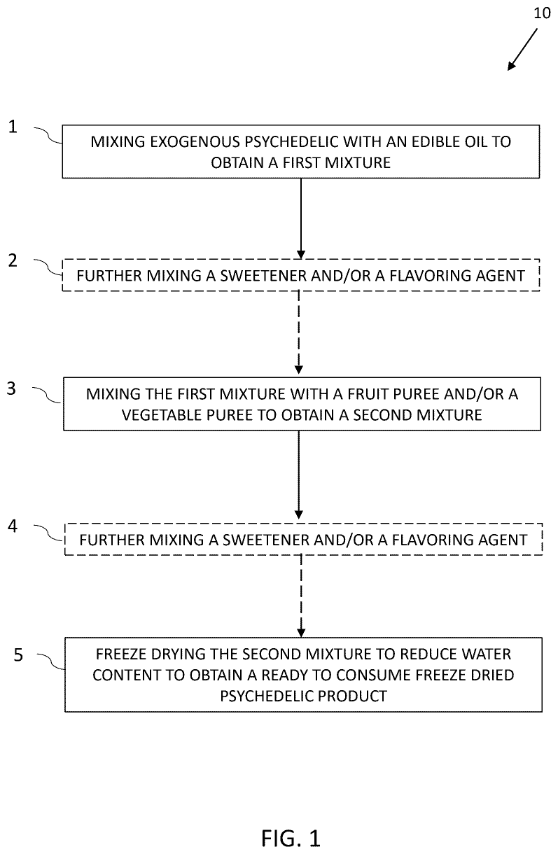

FIG. 1 is a flowchart of steps in a process of preparing a freeze-dried psychedelic product, according to some embodiments.

### DETAILED DESCRIPTION OF EMBODIMENTS

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, there is provided a psychedelic product or composition comprising: at least about 10% w/w of a fruit and/or vegetable puree; at least about 4% w/w of an edible oil; and at least about 0.000001% w/w-about 10% w/w of a psychedelic compound; wherein the psychedelic composition is freeze dried into a ready to be consumed product form which melts in the mouth. According to some embodiments, at least one component of the psychedelic product or composition may be organic.

As used herein, according to some embodiments, the term “organic” may refer to a component produced, grown, or farmed using, while complying with the standards of organic farming. Standards vary worldwide, but organic farming features practices that cycle resources, promote ecological balance, and conserve biodiversity. Organizations regulating organic products may restrict the use of certain pesticides and fertilizers in the farming methods used to produce such products. Organic foods are typically not processed using irradiation, industrial solvents, or synthetic food additive.

As used herein, according to some embodiments, the terms “psychedelic” and “psychedelic compound” may be used interchangeably and may refer to hallucinogenic drugs whose primary effect is to trigger non-ordinary states of consciousness (known as psychedelic experiences or “trips”). Typically, the psychedelics cause specific psychological, visual, and auditory changes, and often a substantially altered state of consciousness. The “classical” psychedelics, the psychedelics with the largest scientific and cultural influence, are mescaline, LSD, psilocybin, MDMA and DMT.

According to some embodiments, the psychedelic compound may be a medicinal and/or recreational compound.

According to some embodiments, the psychedelic compound may be organic. According to some embodiments, the psychedelic compound may be a natural extract or compound, a derivative of a natural compound, a synthetic compound, or a mixture of more than one compound. For example, the psychedelic compound may be psilocin, psilocybin, ketamine, ergoline, lysergamide (e.g., lysergic acid diethylamide (LSD)), methamphetamine (e.g., 3,4-methyl enedioxymethamphetamine (MDMA)), mescaline, ibogaine, tryptamine (e.g., N,N-dimethyltryptamine (DMT)), phenethylamine, N-methoxybenzyl compound, 4-bromo-2,5-dimethoxyphenethylamine, myristicin, benzoxazines, beta-carboline, entheogen, peyote, serotonergic hallucinogen, etc. and/or derivatives, metabolites, isomers, stereoisomers, or combinations thereof. Each possibility is a separate embodiment.

According to some embodiments, the psychedelic compound may be a plant, or part thereof. For example, the plant may be a fungus (e.g., psilocybin mushroom, etc.), cactus (e.g., peyote cactus, etc.), herbaceous plant (e.g., Salvia divinorum, etc.), flowering plant (e.g., various morning-glory species such as Turbina corymbose, datura species, etc.), and/or combinations thereof. Each possibility is a separate embodiment. According to some embodiments, the psychedelic compound may be all or part of a plant or fungus, such as a root, stem, leaf, seed, flower, and/or fruit. Each possibility is a separate embodiment. According to some embodiments, the plant or mushroom may be in the form of all or part of a plant or fungus, fruit body, seed, root, leaf, etc. (e.g., a whole mushroom), pieces or chunks of the plant or fungus, a powder, a dried substance, an extract, a mash, or a liquid.

According to some embodiments, the psychedelic compound may be or include a fungus. According to some embodiments, the fungus may be a psilocybin mushroom.

According to some embodiments, the psychedelic compound may be or include a cactus. According to some embodiments, the cactus may be a peyote cactus.

According to some embodiments, the psychedelic compound may be or include a herbaceous plant. According to some embodiments, the herbaceous plant may be a Salvia divinorum, etc.

According to some embodiments, the psychedelic compound may be or include a flowering plant. According to some embodiments, the flowering plant may be a various morning-glory species such as Turbina corymbose, datura species, etc.

According to some embodiments, the psychedelic compound may be or include a synthetic compound and/or a derivative of a naturally occurring compound.

According to some embodiments, the psychedelic compound may be an extract or concentrate or powder. As a non-limiting example, the psychedelic compound may be a plant extract. According to some embodiments, the psychedelic compound may be a compound extracted from a plant, a derivative or metabolite thereof.

According to some embodiments, the composition may include about 0.000001% to about 50% w/w of the psychedelic compound. According to some embodiments, the composition may include about 0.000002% w/w to about 38% w/w of the psychedelic compound. According to some embodiments, the composition may include about 0.000003% w/w to about 35% w/w of the psychedelic compound. According to some embodiments, the composition may include about 0.000005% w/w to about 35% w/w of the psychedelic compound. According to some embodiments, the composition may include about 0.000005% w/w to about 30% w/w of the psychedelic compound. According to some embodiments, the composition may include about 0.000005% w/w to about 25% w/w of the psychedelic compound. According to some embodiments, the composition may include about 0.000005% w/w to about 20% w/w of the of the psychedelic compound. According to some embodiments, the composition may include about 0.000001% w/w to about 8% w/w, or about 0.000002% w/w to about 7% w/w, or about 0.000003% w/w to about 5% w/w of the psychedelic compound. According to some embodiments, the composition may include about 0.000001% w/w to about 10% w/w, about 0.000001% w/w to about 5% w/w, about 0.000001% w/w to about 1% w/w, about 0.000005% w/w to about 10% w/w, about 0.000006% w/w to about 10% w/w, about 0.000007% w/w to about 10% w/w, or about 0.000008% w/w to about 10% w/w or any other range within the range of 0.00001% w/w to about 10% w/w of the of the psychedelic compound. Each possibility is a separate embodiment. According to some embodiments, the composition may include at least about 0.000001% w/w, at least about 0.005% w/w, at least about 0.000001% w/w, at least about 0.00005% w/w, at least about 0.0005% w/w, at least about 0.005% w/w, at least about 0.05% w/w, at least about 0.5% w/w, at least about 1% w/w, at least about 2% w/w, at least about 5% w/w, at least about 10% w/w, or at least about 15% w/w of the psychedelic compound. Each possibility is a separate embodiment. According to some embodiments, the amount of the psychedelic compound may depend on the psychedelic compound selected for use in the psychedelic composition.

According to some embodiments, the composition may include about 10% w/w, about 15% w/w, about 20% w/w, about 25% w/w, about 30% w/w, about 40% w/w, about 50% w/w, about 60% w/w, about 70% w/w or about 80% w/w of a plant or mushroom which includes a psychedelic compound. Each possibility is a separate embodiment. According to some embodiments, the plant or mushroom may be in the form of part or all of a plant or fungus, fruit body, seed, root, leaf, etc. (e.g., a whole mushroom), pieces or chunks of the plant or fungus, a powder, a dried substance, an extract, a mash, or a liquid. Each possibility is a separate embodiment.

According to some embodiments, the amount of the psychedelic compound in the composition may depend on form of the psychedelic compound.

According to some embodiments, the psychedelic compound may be or may include psilocybin, LSD, MDMA and/or ketamine. Each possibility is a separate embodiment.

According to some embodiments, the composition may comprise about 1-5 g dried psilocybin mushroom or about 10-50 mg isolated psilocybin. According to some embodiments, the composition may comprise about 10 5 w/w-about 70% w/w dried psilocybin mushroom or about 0.001% w/w-about 1% w/w isolated psilocybin.

According to some embodiments, the composition may comprise about 10-400 μg LSD. According to some embodiments, the composition may comprise about 0.00001% w/w-about 0.001% w/w LSD.

According to some embodiments, the composition may comprise about 10-500 mg MDMA. According to some embodiments, the composition may comprise about 0.006% w/w-about 5% w/w MDMA.

According to some embodiments, the composition may comprise about 30-200 mg ketamine. According to some embodiments, the composition may comprise about 0.003% w/w-about 5% w/w ketamine.

According to some embodiments, at least a portion of the psychedelic compound may be encapsulated. According to some embodiments, all the psychedelic compound may be encapsulated. This may advantageously ensure a prolonged and/or gradual release of the psychedelic compound.

According to some embodiments, an initial boost may be released immediately upon consumption (e.g., within 1 min, 30 seconds, 20 seconds or 10 seconds from consumption) by adding un-encapsulated psychedelic compound. Each possibility is a separate embodiment. Such an immediate boost delivery may, for example, be advantageous when urgent treatment is required. According to some embodiments, such an initial boost may begin to take effect within about 10 mins, within about 20 mins, within about 40 mins, or within about 1 hr, Each possibility is a separate embodiment.

According to some embodiments, the encapsulated psychedelic compound may be released gradually and/or over a prolonged period of time e.g., over a period of 1 hour, 2 hours, 3 hours, or 5 hours after consumption of the composition.

According to some embodiments, the capsules may be microcapsules having a diameter of about 1-about 1,000 μm, from about 1-about 500 μm, about 1-about 250 μm, about 5-about 250 μm, or any other range within the about 1-about 1,000 μm range. Each possibility is a separate embodiment. According to some embodiments, the capsules may be nanocapsules having a diameter of less than 1 micron, e.g., about 10-about 999 nm, about 10-about 500 nm, about 10-about 250 nm, about 100-about 250 nm, or any other range within the about 10-999 nm range. Each possibility is a separate embodiment.

According to some embodiments, the capsules may be coated by a functional coating, e.g., a slow release coating, delayed release coating, rapid release coating, immediate release coating, controlled release coating, enteric coating, etc. Each possibility is a separate embodiment.

According to some embodiments, the term “fruit puree” may refer to any type of fruit or combination of fruits that has been cooked, ground, pressed, blended or sieved until it reaches a consistency of a creamy paste or liquid.

According to some embodiments, the term “vegetable puree” may refer to any type of vegetable or combination of vegetables that has been cooked, ground, pressed, blended or sieved until it reaches a consistency of a creamy paste or liquid. Each possibility is a separate embodiment.

According to some embodiments, the composition includes about 15% w/w to about 99% w/w of the fruit and/or vegetable puree. According to some embodiments, the composition includes about 20% w/w to about 60% w/w of the fruit and/or vegetable puree. According to some embodiments, the composition includes at least about 15% w/w, or at least about 20% w/w of the fruit and/or vegetable puree. According to some embodiments, the composition includes about 20% w/w, about 25% w/w, about 28% w/w, about 30% w/w, about 35% w/w, about 40% w/w, about 50% w/w, about 55% w/w, about 65% w/w, about 75 w/w, about 85% w/w, about 95% w/w or about 99% w/w of the fruit and/or vegetable puree. Each possibility is a separate embodiment.

According to some embodiments, the amount of fruit and/or vegetable puree in the composition depends on the psychedelic compound.

As a non-limited example, the composition preferably includes about 15% w/w to about 99% w/w or about 20% w/w to about 60% w/w of the fruit puree. Each possibility is a separate embodiment.

As a non-limited example, the composition preferably includes about 15% w/w to about 99% w/w or about 20% w/w to about 60% w/w of the vegetable puree. Each possibility is a separate embodiment.

According to some embodiments, the composition may in addition to, or as an alternative to the fruit puree include a vegetable puree. Thus, in some embodiments, the composition may include a fruit puree and/or a vegetable puree.

According to some embodiments, the composition is suitable for use as a food product. The term “food product” as used herein should be understood to encompass a product that is consumed to provide nutrition to a subject. The food product is in a solid form that is ready to be consumed by directly introducing to the mouth.

It is further defined that the composition of the present invention is “freeze-dried”, also known as “lyophilization” and “cryodesiccation” resulting in a chewable and/or meltable solid form. As used herein, according to some embodiments, the term “freeze drying” may refer to a low temperature dehydration process that involves freezing the product, lowering pressure, followed by a removing of ice by sublimation. This is in contrast to dehydration by most conventional methods that evaporate water using heat or vacuum (e.g., no less than 7 Mbar) or both.

Additionally, a composition of the present invention is defined to be a “ready to be consumed product”, thus it does not have to be further prepared in any manner. It does not have to be cooked, rehydrated, steamed, mixed with a liquid (hot or cold), fried, microwaved or any other form of preparation.

Further, a composition of the present invention is defined to be a “bite”, which is defined herein as a single serving of a freeze-dried composition which melts in the mouth. According to some embodiments, the bite may weigh between 0.5-15 g or between 1-10 g. According to some embodiments, the bite may be used for oral administration of the psychedelic compound. According to some embodiments, the bite may be used for sublingual of the psychedelic compound. According to some embodiments, the bite may disperse rapidly in the mouth without requiring water. Advantageously, a bite may provide better pre-gastric absorption for psychedelic compounds, protection from counterfeiting and/or more efficient delivery of the psychedelic compound. Additionally, a bite may be ideal for administration to populations which suffer from dysphagia, require fast onset, and ease of use.

According to some embodiments, the composition may be a solid porous matrix (non-powdered). According to some embodiments, the composition may have the form of a single bite. According to some embodiments, the volume of the single bite may be at least about 4 cm³, at least about 5 cm³, at least about 6 cm³, or at least about 8 cm³. Each possibility is a separate embodiment. According to some embodiments, the volume of the single bite may be between about 4 cm³ and about 8 cm³ or about 4 cm³ and about 6 cm³. Each possibility is a separate embodiment.

According to some embodiments, the psychedelic composition is suitable for use in the treatment of a psychiatric disease or disorder. According to some embodiments, the psychiatric disease or disorder include: mood disorders such as depression, major depressive disorder, anxiety, etc., obsessive compulsive disorder (OCD), alcohol-use disorder, drug use disorders, eating disorders such as anorexia nervosa, post-traumatic stress disorder (PTSD), schizophrenia, addiction, etc. Each possibility is a separate embodiment.

As used herein, the term “edible oil” is fat of plant, animal or microbial origin. According to some embodiments, the edible oil is meltable at temperatures below about 40° C., below about 37° C., below about 35° C., below about 30° C. or at about room temperature, and is suitable for food use. Non-limiting examples of suitable oils include coconut oil, palm oil and palm kernel oil, soybean oil, canola oil (rapeseed oil), corn oil, peanut oil, sunflower oil, olive oil and other vegetable oils. Each possibility is a separate embodiment. According to some embodiments, the edible oil is coconut oil. According to some embodiments, the edible oil may be organic.

According to some embodiments, the composition may include about 4% w/w to about 40% w/w of the edible oil. According to some embodiments, the composition includes about 15% w/w to about 30% w/w of the edible oil. According to some embodiments, the composition includes at least about 15% w/w, or at least about 20% w/w of the edible oil. According to some embodiments, the composition includes about 4% w/w, about 10% w/w, about 15% w/w, about 20% w/w, about 25% w/w, about 30% w/w, or about 40% w/w of the edible oil. Each possibility is a separate embodiment.

According to some embodiments, the composition has less than about 5% w/w water. The term “less than about 5% w/w of water” should be understood to relate to the amount of water present in the final food product of the invention. Since the food product of the invention is solid and chewable/meltable, the amount of hydration in the product to be consumed is reduced to less than about 5% by weight of the product.

According to some embodiments, the composition has between about 1% w/w to about 5% w/w of water. In some embodiments, the composition has about 1% w/w, about 2% w/w, about 3% w/w, about 4% w/w, or about 5% w/w. Each possibility is a separate embodiment.

According to some embodiments, the composition may further include a sweetener. According to some embodiments, the sweetener may be organic. According to some embodiments, the sweetener may be a natural sweetener. Non-limiting examples of suitable natural sweeteners include sugar, glucose, fructose, stevia, erythritol, Xylitol, Mannitol, Yacon syrup, honey, maple, molasses, coconut sugar, Monk fruit sweetener and any combination thereof. Each possibility is a separate embodiment. According to some embodiments, the sweetener may be a synthetic sweetener. Non-limiting examples of suitable natural sweeteners include aspartame, Acesulfame potassium, Advantame, Aspartame-acesulfame salt, Cyclamate, Neotame, Neohesperidin, Sacchari, Sucralose or any combination thereof. Each possibility is a separate embodiment.

According to some embodiments, the sweetener is white sugar. According to some embodiments, the white sugar may be organic.

According to some embodiments, the composition may contain no sweetener, e.g., the fruit and/or vegetable puree may require no added sweetener.

According to some embodiments, the composition comprises at least about 0% w/w, at least about 5% w/w, at least about 10% w/w, at least about 15% w/w, at least about 20% w/w, or at least about 25% w/w, or at least about 30% w/w of the white sugar and/or other sweetener. Each possibility is a separate embodiment. According to some embodiments, the composition comprises about 10% w/w to about 40% w/w of white sugar or other sweetener. According to some embodiments, the composition comprises about 15% w/w to about 30% w/w of white sugar or other sweetener. According to some embodiments, the composition comprises about 10% w/w, about 15% w/w, about 20% w/w, about 25% w/w, or about 30% w/w of white sugar and/or other sweetener. Each possibility is a separate embodiment.

In some embodiments, a food product of the invention may further include in addition to, or alternatively to the sweetener, at least about one flavoring agent. According to some embodiments, the flavor agent may be organic. It should be noted that said flavoring agent relates to any type of edible substance added to a product of the invention (at any step of product preparation) that adds and/or alters and/or intensifies the flavor of a product of the invention through the sense of taste and/or smell. In some embodiments said at least about one flavoring agent is a natural flavoring substance (i.e. flavoring substances obtained from plant or animal raw materials, by physical, microbiological or enzymatic processes), a nature-identical flavoring substance (i.e. flavoring substances that are obtained by synthesis or isolated through chemical processes, which are chemically and organoleptically identical to flavoring substances naturally present in products intended for human consumption) or an artificial flavoring substance (i.e. chemically produced flavoring substances that are typically chemically different from the corresponding natural flavoring substance however, in sensory characteristics are the same as natural ones), and any combination thereof.

In some embodiments, said at least about one flavoring agent is selected from the group consisting of a sweetening agent (such as a sweetening saccharide, sugar, sugar substitutes), fructose, galactose, flavoring extracts (such as spices and herb flavors, including vanilla, cinnamon, nutmeg, cardamom and so forth; fruit flavors including banana, strawberry, berry, grape, orange, pineapple, lemon and so forth) and any combinations thereof. Each possibility is a separate embodiment.

According to some embodiments, the food flavoring agent may be a spice or combination of spices, such as, but not limited to: salt, pepper, garlic (for example, in the form of powder), onion (for example, in the form of powder), and the like. According to some embodiments, the food flavoring agent may be an herb or combination of dried spice herbs, such as, but not limited to: oregano, basil, thyme, coriander, parsley, and the like.

According to some embodiments, the flavoring agent may be lemon salt.

According to some embodiments, the composition comprises at least about 0.1% w/w, at least about 0.5% w/w, at least about 1% w/w, at least about 1.5% w/w, at least about 2% w/w, or at least about 3% w/w of lemon salt or other flavoring agent. Each possibility is a separate embodiment. According to some embodiments, the composition comprises about 0.1% w/w to about 5% w/w of lemon salt or other flavoring agent. According to some embodiments, the composition comprises about 1% w/w to about 3% w/w of lemon salt or other flavoring agent. According to some embodiments, the composition comprises about 0.5% w/w, about 1% w/w, about 1.5% w/w, about 2% w/w, or about 2.5% w/w of lemon salt or other flavoring agent. Each possibility is a separate embodiment.

According to some embodiments, the composition may further include a thickening component. According to some embodiments, the thickening component may be organic. In some embodiments, the at least about one thickening component may be a monosaccharide, oligosaccharide (for example disaccharides, like sugar), polysaccharide (for example starch, which can be either native, none-native, modified or processed starch and any combinations thereof) or any combinations thereof. In other embodiments, the at least about one thickening component is starch. In yet further embodiments, the at least about one thickening component is a mixture of starch and sugar. In some embodiments, the at least about one thickening component is starch selected from potato starch, tapioca starch, rice starch, corn starch, wheat starch, maze starch, cassava starch, sweet potato starch or any combinations thereof. Each possibility is a separate embodiment.

In some embodiments, the at least about one thickening component may be between about 10% w/w to about 70% w/w of the product. In some embodiments, the at least about one thickening component is about 10% w/w, about 20% w/w, about 25% w/w, about 30% w/w, about 35% w/w, about 40% w/w, about 45% w/w, about 50% w/w, about 55% w/w, about 60% w/w, about 65% w/w, or about 70% w/w of the product. Each possibility is a separate embodiment.

In some embodiments, a food product of the invention has a mechanical compressibility of at least about 5 N/cm² or about 50 KPa. In some embodiments, a food product of the invention has a mechanical compressibility in the range of between about 5 N/cm² and about 20 N/cm².

In some embodiments, a food product of the invention has a density of at least about 120 mg/cm³. In some embodiments, a food product of the invention has a density in the range of between about 120 mg/cm³ and about 500 mg/cm³.

In some embodiments, the composition is a non-dairy food product, thus it does not contain any dairy component. Alternatively, the composition may further include at least about one dairy component.

According to some embodiments, the composition may further include an additional additive, such as a natural preservative, an oxidating agent, an emulsifier or other food grade agent optimizing the structure, chewability, meltability, preservation or other feature and/or characteristic of the composition. According to some embodiments, such an additional additive may be organic.

The term “a” and “an” refers to one or to more than one (i.e., to at least about one) of the grammatical object of the article. By way of example, “an element” means one element or more than one element.

The term “about” when referring to a measurable value such as an amount, a temporal duration, and the like, is meant to encompass variations of ±20% or in some instances±10%, or in some instances±5%, or in some instances±1%, or in some instances±0.1% from the specified value, as such variations are appropriate to perform the disclosed methods.

According to some embodiments, there is provided a process for the preparation of a freeze-dried psychedelic composition as disclosed herein. Reference is now made to FIG. 1 which shows steps in the preparation process. As shown in FIG. 1, process 10 includes at Step 1, mixing of a psychedelic compound with an edible oil to obtain a first mixture. Step 1 may be performed at a temperature of below about 50° C., below about 40° C., below about 30° C. Step 1 may optionally further include Step 2, mixing a sweetener and/or a flavoring agent in the first mixture. At Step 3, the first mixture obtained in Step 1 is mixed with a selected fruit puree and/or vegetable puree to obtain a second mixture. Step 3 may optionally further include Step 4, mixing a sweetener and/or a flavoring agent with the second mixture. At Step 5, the second mixture obtained in Step 3 is freeze dried under suitable conditions, until water content is reduced to less than about 5% w/w of the final ready to consume psychedelic product.

According to some embodiments, there is further provided a process for the preparation of a freeze-dried psychedelic composition, the process includes the steps of:
a. mixing a psychedelic compound with an edible oil to obtain a first mixture, wherein the mixing is performed at a temperature of below about 50° C., wherein the psychedelic compound may optionally be encapsulated;
b. mixing the first mixture with a fruit and/or vegetable puree to obtain a second mixture;
c. freeze drying the second mixture until water content is less than 5% w/w of the product; thereby producing a freeze-dried psychedelic composition having a ready to be consumed product form, which melts in the mouth.

According to some embodiments, step (a) includes mixing at least about 0.00001% w/w psychedelic compound, at least about 0.000005% w/w psychedelic compound, at least about 0.00007% w/w psychedelic compound, or at least about 0.00009% w/w psychedelic compound with at least about 4% w/w of the edible oil, wherein the weight percent relates to the total weight of the final product.

According to some embodiments, step (b) includes mixing the first mixture with at least about 10% w/w of the fruit puree.

According to some embodiments, the process may further include adding a sweetener and/or flavoring agent at step (a) or (b). According to some embodiments, at least about 15% w/w of sweetener is added, wherein the weight percent relates to the to the total weight of the final product. According to some embodiments, at least about 0.1% w/w flavoring agent may be added in addition to or instead of the sweetener.

According to some embodiments, the second mixture may be cast into a mold prior to freeze drying.

According to some embodiments, the second mixture is frozen and shaped prior to freeze drying.

According to some embodiments, there is provided a method for administering a psychedelic compound to a subject in need thereof, the method comprising inserting into the subject's mouth, the herein disclosed psychedelic composition.

According to some embodiments, the subject is a subject undergoing or having undergone bariatric surgery.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Example 1: Process for the Preparation of a Single Bite Psychedelic Composition

The ingredients (before drying) of the composition are set forth in Table 1 below.

TABLE 1

| Raw Materials | w/w % |
|---|---|
| Fruit and/or vegetable Puree | 15-99 |
| Sweetener | 5-20 |
| Flavoring agent | 0.1-5 |
| Edible oil | 1-15 |
| Psychedelic compound | 0.000001-10 |
| Total 1 Pack | 100 |

Initially, the psychedelic compound is mixed into the edible oil until fully dissolved. The psychedelic compound—edible oil mixture (first mixture) is then mixed with the remaining ingredients to produce a mash-like mixture (second mixture). The mash-like mixture is poured into IQF machine (individual quick freezing), moulds or by depository system, frozen, and subsequently lyophilized until the total amount of water is below 3% w/w, so as to obtain an essentially solid, yet meltable single-dose composition (bite). Preferably, the bite has a weight in the range between about 0.5-15 g, most preferably, the bite has a weight of about 7 g or about 3 g.

### Example 2: Process for the Preparation of One Bite of a Psychedelic Composition Comprising Dried Psilocybin Mushrooms

The ingredients (before drying) of the composition are set forth in Table 2 below.

TABLE 2

| Raw Materials | Weight (g) | w/w % |
|---|---|---|
| Fruit and/or vegetable Puree | 0.7-4.2 | 10-99 |
| White sugar | 0.7-2.1 | 0-20 |
| Lemon salt | 0.07-0.35 | 0.1-5 |
| Coconut oil | 0.7-2.1 | 1-30 |
| Dried psilocybin mushrooms | 1-5 | 10-70 |
| Total 1 Pack | 7.0 | 100 |

Initially, the dried psilocybin mushrooms are mixed into the coconut oil until fully dissolved. The psychedelic-coconut oil mixture (first mixture) is then mixed with the remaining ingredients to produce a mash-like mixture (second mixture). The mash-like mixture is poured into IQF machine (individual quick freezing), moulds or by depository system, frozen, and subsequently lyophilized until the total amount of water is below 3% w/w, so as to obtain an essentially solid, yet meltable single-dose composition.

### Example 3: Process for the Preparation of One Bite of a Psychedelic Composition Comprising Psilocybin

The ingredients (before drying) of the composition are set forth in Table 3 below.

TABLE 3

| Raw Materials | Weight (g) | w/w % |
|---|---|---|
| Fruit and/or vegetable Puree | 1.0-4.9 | 15-99 |
| White sugar | 0.7-2.1 | 1-30 |
| Lemon salt | 0.07-0.35 | 0.1-5 |
| Coconut oil | 0.7-2.1 | 1-30 |
| Psilocybin | 0.007-0.07 | 0.001-1 |
| Total 1 Pack | 7.0 | 100 |

Initially, the psilocybin is mixed into the coconut oil until fully dissolved. The psychedelic-coconut oil mixture (first mixture) is then mixed with the remaining ingredients to produce a mash-like mixture (second mixture). The mash-like mixture is poured into IQF machine (individual quick freezing), moulds or by depository system, frozen, and subsequently lyophilized until the total amount of water is below 3% w/w, so as to obtain an essentially solid, yet meltable single-dose composition.

### Example 4: Process for the Preparation of One Bite of a Psychedelic Composition Comprising LSD

The ingredients (before drying) of the composition are set forth in Table 4 below.

TABLE 4

| Raw Materials | Weight (g) | w/w % |
|---|---|---|
| Fruit and/or vegetable Puree | 1.0-4.9 | 15-99 |
| White sugar | 0.7-2.1 | 0-30 |
| Lemon salt | 0.07-0.35 | 0.1-5 |
| Coconut oil | 0.7-2.1 | 1-30 |
| LSD | 50 μg | <0.01 |
| Total 1 Pack | 7.0 | 100 |

Initially, the LSD is mixed into the coconut oil until fully dissolved. The psychedelic-coconut oil mixture (first mixture) is then mixed with the remaining ingredients to produce a mash-like mixture (second mixture). The mash-like mixture is poured into IQF machine (individual quick freezing), moulds or by depository system, frozen, and subsequently lyophilized until the total amount of water is below 3% w/w, so as to obtain an essentially solid, yet meltable single-dose composition.

### Example 5: Process for the Preparation of One Bite of a Psychedelic Composition Comprising MDMA

The ingredients (before drying) of the composition are set forth in Table 5 below.

TABLE 5

| Raw Materials | Weight (g) | w/w % |
|---|---|---|
| Fruit and/or vegetable Puree | 1.0-4.9 | 15-99 |
| White sugar | 0.7-2.1 | 0-30 |
| Lemon salt | 0.07-0.35 | 0.1-5 |
| Coconut oil | 0.7-2.1 | 1-30 |
| MDMA | 0.07-0.7 | 0.005-10 |
| Total 1 Pack | 7.0 | 100 |

Initially, the MDMA is mixed into the coconut oil until fully dissolved. The psychedelic-coconut oil mixture (first mixture) is then mixed with the remaining ingredients to produce a mash-like mixture (second mixture). The mash-like mixture is poured into IQF machine (individual quick freezing), moulds or by depository system, frozen, and subsequently lyophilized until the total amount of water is below 3% w/w, so as to obtain an essentially solid, yet meltable single-dose composition.

### Example 6: Process for the Preparation of One Bite of a Psychedelic Composition Comprising Ketamine

The ingredients (before drying) of the composition are set forth in Table 6 below.

TABLE 6

| Raw Materials | Weight (g) | w/w % |
|---|---|---|
| Fruit and/or vegetable Puree | 1.0-4.9 | 15-799 |
| White sugar | 0.7-2.1 | 0-30 |
| Lemon salt | 0.07-0.35 | 0.1-5 |
| Coconut oil | 0.7-2.1 | 1-30 |
| Ketamine | 0.005-0.7 | 0.002-5 |
| Total 1 Pack | 7.0 | 100 |

Initially, the ketamine is mixed into the coconut oil until fully dissolved. The psychedelic-coconut oil mixture (first mixture) is then mixed with the remaining ingredients to produce a mash-like mixture (second mixture). The mash-like mixture is poured into IQF machine (individual quick freezing), moulds or by depository system, frozen, and subsequently lyophilized until the total amount of water is below 3% w/w, so as to obtain an essentially solid, yet meltable single-dose composition.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1.-36. (canceled)

37. A freeze-dried psychedelic single composition comprising a mixture of:
(a) a fruit puree and/or a vegetable puree;
(b) an edible oil having a melting temperature of below about 50° C.;
(c) a psychedelic compound; and
(d) less than about 5% w/w water
wherein the psychedelic composition is a freeze-dried bite which melts in the mouth.

38. The composition according to claim 37, wherein the psychedelic compound is a synthetic compound, a natural compound, a derivative of a natural compound, a mineral, an herb, an herb extract, an herb derivative, a fungus, or any combination thereof.

39. The composition according to claim 38, wherein the psychedelic compound comprises psilocybin, lysergic acid diethylamide (LSD), 3,4-methylenedioxy methamphetamine (MDMA) and/or ketamine.

40. The composition according to claim 39, wherein the amount of psilocybin is in the range between about 0.001% w/w-about 1% w/w and wherein the psilocybin is present as dried magic mushrooms in the range between about 10% w/w-about 70% w/w dried psilocybin mushrooms.

41. The composition according to claim 39, wherein the amount of LSD is at least about 0.000001% w/w.

42. The composition according to claim 39, wherein the amount of MDMA is in the range between about 0.005% w/w-about 10% w/w.

43. The composition according to claim 39, wherein the amount of ketamine is in the range between about 0.002% w/w-about 5% w/w.

44. The composition according to claim 37, comprising about 15% w/w-about 99% w/w of the fruit puree and/or vegetable puree.

45. The composition according to claim 37, comprising about 15% w/w-about 70% w/w of a fruit puree or vegetable puree.

46. The composition according to claim 37, further comprising at least about 5% w/w of a sweetener.

47. The composition according to claim 37, comprising about 4% w/w-about 40% w/w of the edible oil.

48. The composition according to claim 37, wherein the edible oil is selected from a vegetable oil, a saturated fatty acid, unsaturated fatty acid, or any combinations thereof.

49. The composition according to claim 37, wherein the psychedelic compound is encapsulated.

50. The composition according to claim 37 comprising a mixture of:
(a) at least about 10% w/w of a fruit puree and/or a vegetable puree;
(b) at least about 4% w/w of an edible oil; and
(c) between about 0.000001% w/w-about 10% w/w of a psychedelic compound.

51. A process for the preparation of a freeze-dried psychedelic composition, the process comprising the steps of:
a. mixing a psychedelic compound with an edible oil to obtain a first mixture, wherein the mixing us performed at a temperature of below about 50° C.;
b. mixing the first mixture with a fruit puree and/or a vegetable puree to obtain a second mixture;
c. freeze drying said second mixture until water content is less than about 5% w/w of the product; thereby producing a freeze-dried psychedelic composition having a ready to be consumed product form, which melts in the mouth.

52. The process according to claim 51, wherein step (a) comprises mixing between about 0.000001% w/w and 10% w/w of the final product of the psychedelic compound with at least about 4% w/w of the final product of the edible oil.

53. The process according to claim 51, wherein step (b) comprises mixing the first mixture with at least about 10% w/w of the final product of the fruit and/or vegetable puree.

54. The process according to claim 51, further comprising adding a sweetener at step (b) and wherein at least about 15% w/w of the final product is the added sweetener.

55. The process according to claim 51, wherein the second mixture is cast into a mold prior to the freeze drying or wherein the second mixture is frozen and shaped prior to freeze drying.

56. A method for treating a psychiatric disorder to a subject in need thereof, the method comprising administering to the patient the composition of claim 37.
